(19) United States
(12) Patent Application Publication
(10) Pub. No.: US 2015/0361392 A1
(43) Pub. Date: Dec. 17, 2015

(54) LIQUID CULTURING OF EPITHELIAL STEM CELLS

(71) Applicant: Genentech, Inc., South San Francisco, CA (US)

(72) Inventor: Brian Biehs, San Francisco, CA (US)

(73) Assignee: GENENTECH, INC., South San Francisco, CA (US)

(21) Appl. No.: 14/835,533

(22) Filed: Aug. 25, 2015

Related U.S. Application Data
(63) Continuation of application No. PCT/US2014/018401, filed on Feb. 25, 2014.
(60) Provisional application No. 61/769,076, filed on Feb. 25, 2013.

(51) Int. Cl. C12N 5/071; A61K 35/38

(52) U.S. Cl. CPC C12N 5/068; A61K 35/38; C12N 2501/155; C12N 2501/415; C12N 2533/90; C12N 2501/11; C12N 2501/727; C12N 2501/42; C12N 2501/148; C12N 2501/115; C12N 2501/13; C12N 2501/117; C12N 2503/02

(57) ABSTRACT

Provided herein is a method of culturing epithelial stem cells and tissue fragments comprising epithelial stem cells in liquid cultures.

### CROSS REFERENCE TO RELATED APPLICATIONS

This application is a continuation of International Application No. PCT/US2014/018401 having an international filing date of Feb. 25, 2014, which claims benefit under 35 U.S.C. §119 to U.S. Patent application No. 61/769,076 filed Feb. 25, 2013, which are hereby incorporated by reference in their entirety.

### FIELD OF THE INVENTION

Provided herein is a method of culturing epithelial stem cells and tissue fragments comprising epithelial stem cells in liquid cultures.

### BACKGROUND

The small intestine epithelium renews every 2 to 5 days, making it one of the most regenerative mammalian tissues. Two types of stem cells have been described in the small intestine based on location and cycling dynamics. See, e.g., Barker et al., Nature 449, 1003-1007 (2007); Sangiorgi, E. & Capecchi, M. R. Nature Genet. 40, 915-920 (2008); Li, L. & Clevers, H. Science 327, 542-545 (2010). Fast-cycling stem cells express markers including Lgr5, Cd133 (also known as Prom1) and Sox9 and are present throughout the intestine. See Zhu, L. et al., Nature 457, 603-607 (2009); Furuyama, K. et al., Nature Genet. 43, 34-41 (2011). Also known as crypt base columnar cells (CBCs), these slender cells populate the crypt and villi within 3 days, and are interspersed among the Paneth cells that support them. See Sato, T. et al., Nature 469, 415-418 (2011); Cheng, H. & Leblond, C. P., Am. J. Anat. 141, 537-561 (1974). Slower-cycling stem cells, marked by enriched expression of Bmi1 or mouse Tert (mTert), represent a rarer cell population. See Sangiorgi, E. & Capecchi, M. R. Nature Genet. 40, 915-920 (2008). These cells form a descending gradient from proximal to distal regions of the intestine, such that they are more prevalent in the duodenum than in the colon. Despite their rarity, Bmi1-expressing stem cells are crucial for crypt maintenance.

A variety of culture systems have been described for culturing primary epithelial stem cells, including intestinal epithelium stem cells (Bjerknes and Cheng, Methods Enzymol 419, 337-83 (2006) and Sato and et al., Nature 459, 262-265 (2009)). To date, the culture systems rely on the use of a solid extracellular matrix to grow the primary epithelial stem cells. Previous work has indicated that the solid extracellular matrix is necessary for maintenance of the pluripotency of epithelial stem and preservation of the basic crypt-villus physiology of crypts that have been isolated from colon or intestine (see e.g., WO2010/090513). The use of an ECM for culturing stem cells has also been shown to enhance long-term survival of the stem cells and the continued presence of undifferentiated stem cells. In the absence of an ECM previously, stem cell cultures could not be cultured for longer periods, and there was no continued presence of undifferentiated stem cells was observed. In addition, the presence of an ECM allowed culturing of three-dimensional tissue organoids, which could not be cultured in the absence of an ECM. However, because solid nature of the culture systems, there are size limitations on the type of test and diagnostic compounds which can be studied using the culture system (e.g., large molecules are unable to diffuse into the solid matrix). Further, as the cells and higher order structures are embedded in the solid matrix, the ease of analysis of the cells and higher order structures is reduced (e.g., the higher order structures (e.g., organoids) must be dissected out of the solid matrix for analysis). Better culture systems of epithelial stem cells, especially intestinal epithelial stem cells, are needed that preserve the physiological structure of maintains the pluripotency of epithelial stem and preserves the basic physiology of an organoid while increasing the types of test and diagnostic compounds and ease of analysis.

### SUMMARY

Provided herein are methods for liquid culturing stem cells. In particular, provided herein are methods for liquid culturing (a) epithelial stem cells and/or (b) isolated epithelial tissue fragments comprising epithelial stem cells, the method comprising incubating the epithelial stem cells and/or the isolated tissue fragments in a liquid cell culture comprising a basal medium for animal or human cells to which is added (i) a Bone Morphogenetic Protein (BMP) inhibitor, (ii) a mitogenic growth factor, (iii) Wnt agonist, and (iv) at least about 4% w/v of extracellular matrix (ECM). Further, provided herein are methods for obtaining and/or growthing a crypt, the method comprising incubating epithelial stem cells and/or isolated tissue fragments in a liquid cell culture comprising a basal medium for animal or human cells to which is added (i) a Bone Morphogenetic Protein (BMP) inhibitor, (ii) a mitogenic growth factor, (iii) Wnt agonist, and (iv) at least about 4% w/v of extracellular matrix (ECM).

In some embodiments of any of the methods, the BMP inhibitor is Noggin, DAN, and/or DAN-like proteins including Cerberus and Gremlin. In some embodiments, the BMP inhibitor is Noggin. In some embodiments, the BMP inhibitor is at a concentration between about 5 and about 500 ng/ml in the liquid cell culture (e.g., about 50 to about 100 ng/mL).

In some embodiments of any of the methods, the Wnt agonist is a Wnt, an R-spondin (RSPO), Norrin, and/or a GSK-inhibitor. In some embodiments, the Wnt agonist is RSPO. In some embodiments, the Wnt agonist is RSPO1. In some embodiments, the Wnt agonist is RSPO2. In some embodiments, the Wnt agonist is RSPO3. In some embodiments, the Wnt agonist is RSPO4. In some embodiments, the Wnt agonist is at a concentration between about 500 ng/mL and about 5 μg/ml in the liquid cell culture (e.g., about 500 to about 1500 ng/mL).

In some embodiments of any of the methods, the mitogenic growth factor is epidermal growth factor (EGF), Transforming Growth Factor-alpha (TGF-α), basic Fibroblast Growth Factor (bFGF), brain-derived neurotrophic factor (BDNF), and Keratinocyte Growth Factor (KGF). In some embodiments, the mitogenic growth factor is EGF. In some embodiments, the mitogenic growth factor is at a concentration between about 5 and about 500 ng/ml in the liquid cell culture (e.g., about 5 to about 50 ng/mL).

In some embodiments of any of the methods, the ECM is a growth factor reduced ECM. In some embodiments, the ECM is matrigel. In some embodiments, the ECM is at a concentration of between about 4% to about 10% w/v in the liquid cell culture. In some embodiments of any of the methods, the method includes culturing in a hanging drop.

In some embodiments of any of the methods, the culture medium further comprises a Rock (Rho-kinase) inhibitor.

In some embodiments of any of the methods, the culture medium further comprises a Notch agonist.

In some embodiments of any of the methods, the epithelial stem cells and/or epithelial tissue fragments are gastrointestinal stem cells and/or gastrointestinal tissue fragments. In some embodiments, the gastrointestinal stem cells and/or gastrointestinal tissue fragments are small intestine stem cells and/or small intestine tissue fragments.

Further provided herein are crypts obtainable by the methods described herein and use of the crypts in a drug discovery screen, toxicity assay, or in regenerative medicine.

### BRIEF DESCRIPTION OF THE FIGURES

FIG. 1A-B. (A) Organoids derived from Lgr5^{DTREGFP} mice exhibit membrane GFP (Lgr5 positive stem cells) and Lysozyme staining (Paneth cells, arrow) in crypt-like structures. (B) Optical cross section.

FIG. 2A-D. Organoids derived from Lgr5^{DTREGFP} mice were cultured in the presence (B, B-1, D) or absence (A, A-1, C) of diptheria toxin (DT) for 10 days. Crypt-like structures and Lysozyme positive cells are preserved in DT treated organoids (B, B-1). Organoids continued to proliferate in the presence of DT (D).

FIG. 3A-D. Organoids derived from Lgr5^{DTREGFP} mice were cultured in various concentrations of Matrigel.

### DETAILED DESCRIPTION

### I. Definitions

The terms “polypeptide” refer herein to a native sequence polypeptide, polypeptide variants and fragments of a native sequence polypeptide and polypeptide variants (which are further defined herein). The polypeptide described herein may be that which is isolated from a variety of sources, such as from human tissue types or from another source, or prepared by recombinant or synthetic methods.

A “native sequence polypeptide” comprises a polypeptide having the same amino acid sequence as the corresponding polypeptide derived from nature.

“Polypeptide variant”, or variations thereof, means a polypeptide, generally an active polypeptide, as defined herein having at least about 80% amino acid sequence identity with any of the native sequence polypeptide sequences as disclosed herein. Such polypeptide variants include, for instance, polypeptides wherein one or more amino acid residues are added, or deleted, at the N- or C-terminus of a native amino acid sequence. Ordinarily, a polypeptide variant will have at least about 80% amino acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% amino acid sequence identity, to a native sequence polypeptide sequence as disclosed herein. Ordinarily, variant polypeptides are at least about 10 amino acids in length, alternatively at least about 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600 amino acids in length, or more. Optionally, variant polypeptides will have no more than one conservative amino acid substitution as compared to a native polypeptide sequence, alternatively no more than 2, 3, 4, 5, 6, 7, 8, 9, or 10 conservative amino acid substitution as compared to the native polypeptide sequence.

An “isolated” refers to a polypeptide, antibody, nucleic acid, etc. which has been separated from a component of its natural environment. In some embodiments for an antibody, the antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, can be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) overnight hybridization in a solution that employs 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with a 10 minute wash at 42° C. in 0.2×SSC (sodium chloride/sodium citrate) followed by a 10 minute high-stringency wash consisting of 0.1×SSC containing EDTA at 55° C.

“Moderately stringent conditions” can be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

By “tissue sample” or “tissue fragments” is meant a collection of similar cells obtained from a tissue of a subject or individual. The source of the tissue sample or tissue fragments may be solid tissue as from a fresh, frozen and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. tissue sample or tissue fragments may also be primary or cultured cells or cell lines. Optionally, the tissue sample or tissue fragments is obtained from a disease tissue/organ. The tissue sample may contain compounds which are not naturally intermixed with the tissue in nature such as preservatives, anticoagulants, buffers, fixatives, nutrients, antibiotics, or the like.

A “reference sample”, “reference cell”, “reference tissue”, “control sample”, “control cell”, or “control tissue”, as used herein, refers to a sample, cell, tissue, standard, or level that is used for comparison purposes. In one embodiment, a reference sample, reference cell, reference tissue, control sample, control cell, or control tissue is obtained from a healthy and/or non-diseased part of the body (e.g., tissue or cells) of the same subject or individual. For example, healthy and/or non-diseased cells or tissue adjacent to the diseased cells or tissue (e.g., cells or tissue adjacent to a tumor). In another embodiment, a reference sample is obtained from an untreated tissue and/or cell of the body of the same subject or individual. In yet another embodiment, a reference sample, reference cell, reference tissue, control sample, control cell, or control tissue is obtained from a healthy and/or non-diseased part of the body (e.g., tissues or cells) of an individual who is not the subject or individual. In even another embodiment, a reference sample, reference cell, reference tissue, control sample, control cell, or control tissue is obtained from an untreated tissue and/or cell of the body of an individual who is not the subject or individual.

The term “substantially the same,” as used herein, denotes a sufficiently high degree of similarity between two numeric values, such that one of skill in the art would consider the difference between the two values to be of little or no biological and/or statistical significance within the context of the biological characteristic measured by the values (e.g., Kd values or inhibition). The difference between the two values is, for example, less than about 50%, less than about 40%, less than about 30%, less than about 20%, and/or less than about 10% as a function of the reference/comparator value.

The phrase “substantially different,” as used herein, denotes a sufficiently high degree of difference between two numeric values such that one of skill in the art would consider the difference between the two values to be of statistical significance within the context of the biological characteristic measured by the values (e.g., Kd values or inhibition). The difference between the two values is, for example, greater than about 10%, greater than about 20%, greater than about 30%, greater than about 40%, and/or greater than about 50% as a function of the value for the reference/comparator molecule.

An “effective amount” of an agent refers to an amount effective, at dosages and for periods of time necessary, to achieve the desired therapeutic or prophylactic result.

A “patient,” an “individual,” or a “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the patient, individual, or subject is a human.

By “reduce or inhibit” is meant the ability to cause an overall decrease of 20%, 30%, 40%, 50%, 60%, 70%, 75%, 80%, 85%, 90%, 95%, or greater. Reduce or inhibit can refer to the symptoms of the disorder being treated, the presence or size of metastases, or the size of the primary tumor.

As is understood by one skilled in the art, reference to “about” a value or parameter herein includes (and describes) embodiments that are directed to that value or parameter per se. For example, description referring to “about X” includes description of “X”.

It is understood that aspect and embodiments of the invention described herein include “consisting” and/or “consisting essentially of” aspects and embodiments. As used herein, the singular form “a”, “an”, and “the” includes plural references unless indicated otherwise.

### II. Methods and Uses

Provided herein are methods for liquid culturing stem cells. Provided herein are methods of culturing of epithelial stem cells and isolated fragments from the small intestine, colon, stomach and pancreas comprising epithelial stem cells in liquid, while preserving the presence of stem cells that retain an undifferentiated phenotype and self-maintenance capabilities. For example, isolated crypts that are cultured according to methods described herein develop into crypt-villus organoids, comprising a central lumen lined by a villus-like epithelium. The resulting organoids undergo multiple crypt fission events. Surprisingly, the methods provided herein allows for the outgrowth of single, isolated epithelial stem cells into crypt-villus organoids in liquid culture in the presence of the low levels of extracellular matrix. Isolated gastric fragments from the pyloric region of the stomach behaved as intestinal crypt organoids: the opened upper part of the unit was sealed, the lumen was filled with apoptotic cells, and the organoids underwent continuous budding events (reminiscent of gland fission) while maintaining their polarity with a central lumen in the low levels of extracellular matrix.

In particular, provided herein are methods for liquid culturing (a) epithelial stem cells and/or (b) isolated epithelial tissue fragments comprising epithelial stem cells, the method comprising incubating the epithelial stem cells and/or the isolated tissue fragments in a liquid cell culture comprising a basal medium for animal or human cells to which is added (i) a Bone Morphogenetic Protein (BMP) inhibitor, (ii) a mitogenic growth factor, (iii) Wnt agonist, and (iv) at least about 4% w/v of extracellular matrix (ECM). Further, provided herein are methods for obtaining and/or growthing a crypt, the method comprising incubating epithelial stem cells and/or isolated tissue fragments in a liquid cell culture comprising a basal medium for animal or human cells to which is added (i) a Bone Morphogenetic Protein (BMP) inhibitor, (ii) a mitogenic growth factor, (iii) Wnt agonist, and (iv) at least about 4% w/v of extracellular matrix (ECM).

Stem cells are found in many organs of adult animals and retain an undifferentiated phenotype, their offspring can differentiate towards all lineages present in the pertinent tissue, they retain self-maintenance capabilities throughout life, and they are able to regenerate the pertinent tissue after injury Stem cells reside in a specialized location, the stem cell niche, which supplies the appropriate cell-cell contacts and signals for maintenance of the stem cell population.

Epithelial stem cells are able to form the distinct cell types of which the epithelium is composed. Some epithelia, such as skin or intestine, show rapid cell turnover, indicating that the residing stem cells must be continuously proliferating. Other epithelia, such as the liver or pancreas, show a very slow turnover under normal conditions. Crypts can be isolated from the duodenum, small and large intestine, including jejunum, ileum, and colon, and the pyloric region of the stomach by protocols that are known to the skilled person. For example, crypts can be isolated by incubation of isolated tissue with chelating agents that release cells from their calcium- and magnesium-dependent interactions with the basement membrane and stromal cell types. After washing the tissue, the epithelial cell layer is scraped from the submucosa with a glass slide and minced. This is followed by incubation in trypsin or, more preferred, EDTA and/or EGTA and separation of undigested tissue fragments and single cells from crypts using, for example, filtration and/or centrifugations steps. Other proteolytic enzymes, such as collagenase and/or dispase I, can be used instead of trypsin. Similar methods are used to isolate fragments of the pancreas and stomach.

Methods to isolate stem cells from epithelial tissue are known in the art. In some embodiments, the method comprises isolating stem cells express Lgr 5 and/or Lgr 6 on their surface, which belong to the large G protein-coupled receptor (GPCR) superfamily. In some embodiments, the method comprises preparing a cell suspension from the epithelial tissue, contacting the cell suspension with an Lgr5 and/or 6 binding compound, isolating the Lgr5 and/or 6 binding compound, and isolating the stem cells from the binding compound. In some embodiments, single cell suspensions comprising epithelial stem cells may be mechanically generated from the isolated crypts.

In some embodiments, the Lgr5 and/or 6 binding compounds comprise antibodies, such as monoclonal antibodies that specifically recognize and bind to the extracellular domain of either Lgr5 or Lgr6, such as monoclonal antibodies including mouse and rat monoclonal antibodies. Using such an antibody, Lgr5 and/or Lgr6-expressing stem cells can be isolated, for example with the aid of magnetic beads or through fluorescence-activated cell sorting. In some embodiments, the epithelial stem cells are isolated from the crypts, gastric fragments or pancreatic fragments. For example, the epithelial stem cells are isolated from crypts that are isolated from the bowel. In some embodiments, the epithelial stem cells are isolated from the small intestine, including duodenum, jejunum and ileum, pancreas or stomach.

In some embodiments of any of the methods, the epithelial stem cells and/or epithelial tissue fragments are gastrointestinal stem cells and/or gastrointestinal tissue fragments. In some embodiments, the gastrointestinal stem cells and/or gastrointestinal tissue fragments are small intestine stem cells and/or small intestine tissue fragments.

A cellular niche is in part determined by the stem cells and surrounding cells, and the extracellular matrix (ECM) that is produced by the cells in the niche. In some embodiments, isolated crypts or epithelial stem cells are attached to an ECM. ECM is composed of a variety of polysaccharides, water, elastin, and glycoproteins, wherein the glycoproteins comprise collagen, entactin (nidogen), fibronectin, and laminin. ECM is secreted by connective tissue cells. Different types of ECM are known, comprising different compositions including different types of glycoproteins and/or different combination of glycoproteins. The ECM can be provided by culturing ECM-producing cells, such as for example fibroblast cells, in a receptacle, prior to the removal of these cells and the addition of isolated crypts or epithelial stem cells. Examples of extracellular matrix-producing cells are chondrocytes, producing mainly collagen and proteoglycans, fibroblast cells, producing mainly type IV collagen, laminin, interstitial procollagens, and fibronectin, and colonic myofibroblasts producing mainly collagens (type I, III, and V), chondroitin sulfate proteoglycan, hyaluronic acid, fibronectin, and tenascin-C Alternatively, the ECM is commercially provided. Examples of commercially available extracellular matrices are extracellular matrix proteins (Invitrogen) and Matrigel™ (BD Biosciences).

In some embodiments, the ECM comprises at least two distinct glycoproteins, such as two different types of collagen or a collagen and laminin The ECM can be a synthetic hydrogel extracellular matrix or a naturally occurring ECM A most preferred ECM is provided by Matrigel™ (BD Biosciences), which comprises laminin, entactin, and collagen IV.

In some embodiments of any of the methods, the ECM is a growth factor reduced ECM. In some embodiments, the ECM is matrigel.

In some embodiments of any of the methods, the ECM is at a concentration of between about any of about 4% to about 10%, about 4% to about 5%, and/or about 4% to about 15%, w/v in the liquid cell culture. In some embodiments, the ECM is at a concentration greater than about any of 4%, 5%, 6%, 7%, 8%, 9%, and/or 10% and less than about any of 20%, 19%, 18%, 17%, 16%, 15%, 14%, 13%, 12%, 11%, and/or 10%. In some embodiments, the ECM is at a concentration of about any of 4%, 5%, 6%, 7%, 8%, 9%, and/or 10%.

A cell culture medium that is used in a methods described herein may comprise any cell culture medium. In some embodiments, the cell culture medium is a defined synthetic medium that is buffered at a pH of 7.4 (e.g., between 7.2 and 7 6 or at least 7.2 and not higher than 7.6) with a carbonate-based buffer, while the cells are cultured in an atmosphere comprising between 5% and 10% CO₂, or at least 5% and not more than 10% CO₂, preferably 5% CO₂. In some embodiments, the cell culture medium is selected from DMEM/F12 and RPMI 1640 supplemented with glutamine, insulin, Penicillin/streptomycin and transferrin. In some embodiments, Advanced DMEM/F12 or Advanced RPMI is used, which is optimized for serum free culture and already includes insulin In this case, the Advanced DMEM/F12 or Advanced RPMI medium is preferably supplemented with glutamine and Penicillin/streptomycin. In some embodiments, the cell culture medium is supplemented with a purified, natural, semi-synthetic and/or synthetic growth factor and does not comprise an undefined component such as fetal bovine serum or fetal calf serum. Supplements such as, for example, B27 (Invitrogen), N-Acetylcysteine (Sigma) and N2 (Invitrogen) stimulate proliferation of some cells and may further be added to the medium.

In some embodiments, the basal culture media comprises a BMP inhibitor. BMPs bind as a dimeric ligand to a receptor complex consisting of two different receptor serine/threonine kinases, type I and type II receptors. The type II receptor phosphorylates the type I receptor, resulting in the activation of this receptor kinase. The type I receptor subsequently phosphorylates specific receptor substrates (SMAD), resulting in a signal transduction pathway leading to transcriptional activity.

A BMP inhibitor, in some embodiments, is an agent that binds to a BMP molecule to form a complex wherein the BMP activity is neutralized, for example by preventing or inhibiting the binding of the BMP molecule to a BMP receptor. Alternatively, the inhibitor is an agent that acts as an antagonist or reverse agonist. This type of inhibitor binds with a BMP receptor and prevents binding of a BMP to the receptor. An example of a latter agent is an antibody that binds a BMP receptor and prevents binding of BMP to the antibody-bound receptor.

Several classes of natural BMP-binding proteins are known, including Noggin (Peprotech), Chordin and chordin-like proteins (R&D systems) comprising chordin domains, Follistatin and follistatin-related proteins (R&D systems) comprising a follistatin domain, DAN and DAN-like proteins (R&D systems) comprising a DAN cysteine-knot domain, sclerostin/SOST (R&D systems), decorin (R&D systems), and alpha-2 macroglobulin (R&D systems). In some embodiments, BMP inhibitor is selected from Noggin, DAN, and DAN-like proteins including Cerberus and Gremlin (R&D systems). In some embodiments, the BMP inhibitor is Noggin. In some embodiments, the diffusible proteins are able to bind a BMP ligand with varying degrees of affinity and inhibit their access to signaling receptors. The addition of any of these BMP inhibitors to the basal culture medium prevents the loss of stem cells, which otherwise occurs after about 2-3 weeks of culture.

In some embodiments, the BMP inhibitor inhibits a BMP-dependent activity in a cell to at most 90%, at most 80%, at most 70%, at most 50%, at most 30%, at most 10%, or 0%, relative to a level of a BMP activity in the absence of the inhibitor. In some embodiments, a BMP activity can be determined by measuring the transcriptional activity of BMP, for example as exemplified in Zilberberg et al, 2007 BMC Cell Biol 8:41.

In some embodiments, the BMP inhibitor is at a concentration between about any of 5 and 500 ng/mL, 5 and 250 ng/mL, 25 and 150 ng/mL, and 50 and 100 ng/mL in the liquid cell. In some embodiments, the BMP inhibitor in the basal cell culture is at a concentration of at least about any of 10 ng/ml, 20 ng/ml, 50 ng/ml, 100 ng/ml. In some embodiments, the concentration of BMP inhibitor is about 100 ng/ml. During culturing of stem cells, the BMP inhibitor is preferably added to the culture medium every second day, while the culture medium is refreshed preferably every fourth day. In some embodiments, the BMP inhibitor is Noggin.

In some embodiments of any of the methods, a Wnt agonist is added to the basal culture medium. The Wnt signaling pathway is defined by a series of events that occur when a Wnt protein binds to a cell-surface receptor of a Frizzled receptor family member. This results in the activation of Dishevelled family proteins which inhibit a complex of proteins that includes axin, GSK-3, and the protein APC to degrade intracellular β-catenin. The resulting enriched nuclear β-catenin enhances transcription by TCF/LEF family transcription factors. In some embodiments, Wnt agonist may be an agent that activates TCF/LEF-mediated transcription in a cell. Wnt agonists are therefore selected from true Wnt agonists that bind and activate a Frizzled receptor family member including any and all of the Wnt family proteins, an inhibitor of intracellular β-catenin degradation, and activators of TCF/LEF.

A Wnt agonist comprises a secreted glycoprotein including, but not limited to Wnt-1/Int-1, Wnt-2/Irp (e.g., InM-related Protein), Wnt-2b/13, Wnt-3/Int-4, Wnt-3a (e.g., R&D systems), Wnt-4, Wnt-5a, Wnt-5b, Wnt-6 (e.g., Kirikoshi H et al. (2001) Biochem Biophys Res Com 283:798-805), Wnt-7a (e.g., R&D systems). Wnt-7b, Wnt-8a/8d, Wnt-8b, Wnt-9a/14, Wnt-9b/14b/15, Wnt-10a, Wnt-10b/12, WnM 1, and/or Wnt-16. An overview of human Wnt proteins is provided in “THE WNT FAMILY OF SECRETED PROTEINS”, R&D Systems Catalog, 2004. In some embodiments, the Wnt agonist is a Wnt family member, R-spondin 1-4, Norrin, and/or a GSK-inhibitor.

In some embodiments, the Wnt agonist is an R-spodin polypeptide. R-spondin family of secreted proteins are implicated in the activation and regulation of Wnt signaling pathway and comprise 4 members (R-spondin 1 (e.g., NU206, Nuvelo, San Carlos, Calif.), R-spondm 2 (e.g., R&D systems), R-spondin 3, and R-spondin-4).

In some embodiments, the Wnt agonist is Norrin (also called Nome Disease Protein or NDP) (e.g., R&D systems)), which is a secreted regulatory protein that functions like a Wnt protein in that it binds with high affinity to the Frizzled-4 receptor and induces activation of the Wnt signaling pathway (Kestutis Planutis et al. (2007) BMC Cell Biol 8:12). A small-molecule agonist of the Wnt signaling pathway, an aminopyrimidine derivative, was recently identified and is also expressly included as a Wnt agonist (Lin et al. (2005) Angew Chem Int Ed Engl 44:1987-90).

In some embodiments, the Wnt agonist is a GSK-inhibitor. Examples of GSK-inhibitors include, but are not limited to, small-interfering RNAs (siRNA, e.g., Cell Signaling), lithium (e.g., Sigma), kenpaullone (Biomol International, Leost et al. (2000) Eur J Biochem 267, 5983-5994), 6-Bromoindirubin-30-acetoxime (Meyer et al. (2003) Chem Biol 10, 1255-1266), SB 216763 and SB 415286 (Sigma-Aldrich), and FRAT-family members and FRAT-derived peptides that prevent interaction of GSK-3 with axin (Meijer et al. (2004) Trends in Pharma. Sci. 25, 471-480), which are hereby incorporated by reference. Methods and assays for determining a level of GSK-3 inhibition are known to a skilled person and comprise, for example, the methods and assay as described in Liao et al. (2004) Endocrinology, 145(6) 2941-9.

In some embodiments, the Wnt agonist stimulates a Wnt activity in a cell by at least 10%, at least 20%, at least 30%, at least 50%, at least 70%, at least 90%, at least 100%, relative to a level of the Wnt activity in the absence of the molecule As is known to a skilled person, a Wnt activity can be determined by measuring the transcriptional activity of Wnt, for example by pTOPFLASH and pFOPFLASH Tcf luciferase reporter constructs (Korinek et al. (1997) Science 275 1784-1787).

In some embodiments, the Wnt agonist is at a concentration between about any of 500 ng/mL and 5 μg/ml, 500 ng/mL and 5 μg/mL, 500 ng/mL and about 1.5 μg/mL in the liquid cell culture (e.g., about 500 to about 1500 ng/mL). In some embodiments, Wnt agonist is added to the basal culture medium at a concentration of at least about any of 50 ng/mL, 100 ng/mL, 200 ng/mL, 300 ng/mL, 500 ng/mL, 750 ng/mL, 1000 ng/mL, 1250 ng/mL, and/or 1500 ng/mL. In some embodiments, the concentration of Wnt agonist is about 500 ng/ml. During culturing of stem cells, the Wnt agonist is preferably added to the culture medium every second day, while the culture medium is refreshed preferably every fourth day. In some embodiments, the Wnt agonist comprises or consists of R-spondin 1. In some embodiments, the Wnt agonist comprises or consists of R-spondin 2. In some embodiments, the Wnt agonist comprises or consists of R-spondin 3. In some embodiments, the Wnt agonist comprises or consists of R-spondin 4.

In some embodiments, the Wnt agonist is selected from the group consisting of R-spondin, Wnt-3a and Wnt-6. In some embodiments, R-spondin and Wnt-3a are both used as Wnt agonist. In some embodiments, the concentrations are about 500 ng/ml for R-spondin and about 100 ng/ml for Wnt3a.

In some embodiments, the basal culture medium comprises a mitogenic growth factor. Example of mitogen growth factors include, but are not limited to, epidermal growth factor (EGF, e.g., Peprotech), Transforming Growth Factor-alpha (TGF-alpha, e.g., Peprotech), basic Fibroblast Growth Factor (bFGF, e.g., Peprotech), brain-derived neurotrophic factor (BDNF, R&D Systems), and Keratinocyte Growth Factor (KGF, Peprotech). EGF is a potent mitogenic factor for a variety of cultured ectodermal and mesodermal cells and has a profound effect on the differentiation of specific cells in vivo and in vitro and of some fibroblasts in cell culture. The EGF precursor exists as a membrane-bound molecule which is proteolytically cleaved to generate the 53-amino acid peptide hormone that stimulates cells.

In some embodiments, mitogenic growth factor is added to the basal culture medium at a concentration of between 5 and 500 ng/ml or of at least 5 and not higher than 500 ng/ml. In some embodiments, the concentration is at least about any of 5, 10, 20, 25, 30, 40, 45, or 50 ng/mL and not higher than about any of 500, 450, 400, 350, 300, 250, 200, 150, or 100 ng/mL. In some embodiments, the concentration of the mitogenic growth factor is at least about 50 and not higher than about 100 ng/ml. In some embodiments, the concentration is about 50 ng/ml. In some embodiments, the mitogenic growth factor is EGF. In some embodiments, the mitogenic growth factor is bFGF, (e.g., FGF10 or FGF7). In some embodiments, FGF7 and/or FGF10 is used. FGF7 is also known as KGF (Keratinocyte Growth Factor.) In some embodiment, a combination of mitogenic growth factors such as, for example, EGF and KGF, or EGF and BDNF, is added to the basal culture medium. In some embodiments, a combination of mitogenic growth factors such as, for example, EGF and KGF, or EGF and FGF10, is added to the basal culture medium. If more than one mitogenic growth factor is used, for example FGF7 and FGF10, the concentration of a mitogen growth factor is as defined above and refers to the total concentration of mitogen growth factor used. In some embodiments during culturing of stem cells, the mitogenic growth factor is added to the culture medium every second day, while the culture medium is refreshed every fourth day. Any member of the bFGF family may be used.

In some embodiments, the culture medium comprises a Rock (Rho-kinase) inhibitor. The addition of a Rock inhibitor was found to prevent anoikis, especially when cultering single stem cells The Rock inhibitor is preferably selected from R)-(+)-trans-4-(1-aminoethyl)-N-(4-Pyridyl)cyclohexanecarboxamide dihydrochloride monohydrate (Y-27632, e.g., Sigma-Aldrich), 5-(1,4-diazepan-1-ylsulfonyl)isoquinoline (fasudil or HA1077, e.g., Cayman Chemical), and (S)-(+)-2-methyl-1-[(4-methyl-5-isoquinolinyl)sulfonyl]-hexahydro-1H-1,4-diazepine dihydrochloride (H-1 152, e.g., Tocris Bioschience). The Rho-kinase inhibitor, for example Y-27632, may be added to the culture medium every second day during the first seven days of culturing the stem cells. In some embodiments, the concentration for Y27632 is 10 DM.

In some embodiments, the culture medium comprises a Notch agonist. Notch signaling has been shown to play an important role in cell-fate determination, as well as in cell survival and proliferation. Notch receptor proteins can interact with a number of surface-bound or secreted ligands, including but not limited to Delta 1, Jagged 1 and 2, and Delta-like 1, Delta-like 3, Delta-like 4. Upon ligand binding, Notch receptors are activated by serial cleavage events involving members of the ADAM protease family, as well as an intramembranous cleavage regulated by the gamma secretase presinilin. The resultant is a translocation of the intracellular domain of Notch to the nucleus where it transcriptionally activates downstream genes. In some embodiments, the Notch agonist is selected from Jagged 1 and Delta 1, or an active fragment or derivative thereof. In some embodiments, Notch agonist is DSL peptide (Dontu et al., 2004. Breast Cancer Res 6:R605-R615) with the sequence CDDYYYGFGCNKFCRPR. The DSL peptide (ANA spec) is preferably used at a concentration between about 10 μM and about 100 nM or at least about 10 μM and not higher than about 100 nM. The addition of a Notch agonist, especially during the first week of culturing, may increase the culture efficiency by a factor of 2-3. In some embodiments, the Notch agonist is added to the culture medium every second day during the first seven days of culturing the stem cells.

In some embodiments, a Notch agonist may be a molecule that stimulates a Notch activity in a cell by at least about any of 10%, 20%, 30%, 50%, 70%, 90%, and/or 100%, relative to a level of a Notch activity in the absence of the molecule. In some embodiments, a Notch activity can be determined by measuring the transcriptional activity of Notch, for example by a 4xwtCBF1-luciferase reporter construct as described (Hsieh et al. (1996) Mol Cell. Biol. 16, 952-959).

In some embodiments of any of the methods, the method includes culturing in a hanging drop. In some embodiments, the method includes culturing in a conventional hanging drop. In some embodiments, the method includes culturing in a hallow sphere hanging drop (Lee et al. Tissue Engineering 15(00) (2009)). In some embodiments of any of the methods, the method includes culturing in a scaffold-free environment. In some embodiments, the method includes culturing in a hanging drop plate (e.g., as described in US2011/0306122 and/or EP2342317, which is incorporated by reference in its entirety).

In some embodiments, the epithelial stem cells are pancreas, stomach, intestinal, and/or colonic epithelial stem cells. In some embodiments, the epithelial stem cells are small intestinal stem cells. In some embodiments, the epithelial stem cells do not comprise embryonic stem cells. In some embodiments, the epithelial stem cells comprise adult stem cells. In some embodiments, the single sorted epithelial stem cells from the small intestine, colon, and stomach are also able to initiate these 3-dimensional organoids in liquid culture.

In some embodiments, the liquid culture methods described herein allows the establishment of long-term culture conditions under which single crypts undergo multiple crypt fission events, while simultaneously generating villus-like epithelial domains in which all differentiated cell types are present. In some embodiments, the cultured crypts undergo dramatic morphological changes after taking them into culture. In some embodiments, the upper opening of freshly isolated crypts becomes sealed and this region gradually balloons out and becomes filled with apoptotic cells, much like apoptotic cells are pinched off at the villus tip. In some embodiments, the crypt region was found to undergo continuous budding events which create additional crypts, a process reminiscent of crypt fission. In some embodiments, the crypt-like extensions comprise all differentiated epithelial cell types, including proliferative cells, Paneth cells, enterocytes and goblet cells. In some embodiments, myofibroblasts or other non-epithelial cells are not detectable in the organoids at any stage.

In some embodiments, the liquid culture methods described herein herein allow expansion of the budding crypt structures to created organoids, comprising >40 crypt-like structures surrounding a central lumen lined by a villus-like epithelium and filled with apoptotic cell bodies. In some embodiments, the crypt-villus organoids comprise a central lumen lined by a villus-like epithelium. In some embodiments, lumen is opened at consecutive time intervals to release the content into the medium. In some embodiments, the liquid culture methods allow culture periods of at least seven months, at least eight months, at least nine months, at least ten months. In some embodiments, the organoids can be passaged and maintained in culture for at least 6 months without losing the essential characteristics. In some embodiment, passaging does involve and/or require manual fragmentation of organoids.

In one aspect, the invention therefore provides crypt-villus organoids, comprising a central lumen lined by a villus-like epithelium that result from culturing of epithelial stem cells or isolated crypts in a culture medium described herein and/or obtainable using a method described herein. In some embodiments, the organoid is a gastric organoid.

For high-throughput purposes, the crypt-villus organoids are cultured in multiwell plates such as. for example, 96 well plates or 384 well plates Libraries of molecules are used to identify a molecule that affects the organoids. Preferred libraries comprise antibody fragment libraries, peptide phage display libraries, peptide libraries (e g LOPAP™, Sigma Aldrich), lipid libraries (BioMol), synthetic compound libraries (e g LOP AC™, Sigma Aldrich) or natural compound libraries (Specs, TimTec). These genetic libraries comprise cDNA libraries, antisense libraries, and siRNA or other non-coding RNA libraries. The cells are preferably exposed to multiple concentrations of a test agent for certain period of time. At the end of the exposure period, the cultures are evaluated. The term “affecting” is used to cover any change in a cell, including, but not limited to, a reduction in, or loss of, proliferation, a morphological change, and cell death. The crypt-villus, gastric or pancreatic organoids can also be used to identify drugs that specifically target epithelial carcinoma cells, but not the crypt-villus, gastric or pancreatic organoids.

The crypt-villus organoids can further replace the use of cell lines such as Caco-2 cells in toxicity assays of potential novel drugs or of known or novel food supplements.

Furthermore, the crypt-villus organoids can be used for culturing of a pathogen such as a norovirus which presently lacks a suitable tissue culture or animal model.

In some embodiments, gene therapy can additionally be used in a method directed at repairing damaged or diseased tissue. Use can, for example, be made of an adenoviral or retroviral gene delivery vehicle to deliver genetic information, like DNA and/or RNA to stem cells A skilled person can replace or repair particular genes targeted in gene therapy. For example, a normal gene may be inserted into a nonspecific location within the genome to replace a nonfunctional gene. In another example, an abnormal gene sequence can be replaced for a normal gene sequence through homologous recombination. Alternatively, selective reverse mutation can return a gene to its normal function A further example is altering the regulation (the degree to which a gene is turned on or off) of a particular gene. Preferably, the stem cells are ex vivo treated by a gene therapy approach and are subsequently transferred to the mammal, preferably a human being in need of treatment.

In some, amino acid sequence variants of the polypeptides (e.g., BMP inhibitors, Wnt agonists, etc.) provided herein are contemplated. For example, it may be desirable to improve the binding affinity and/or other biological properties of the antibody and/or binding polypeptide. Amino acid sequence variants of an antibody and/or binding polypeptides may be prepared by introducing appropriate modifications into the nucleotide sequence encoding the antibody and/or binding polypeptide, or by peptide synthesis. Such modifications include, for example, deletions from, and/or insertions into and/or substitutions of residues within the amino acid sequences of the antibody and/or binding polypeptide. Any combination of deletion, insertion, and substitution can be made to arrive at the final construct, provided that the final construct possesses the desired characteristics, e.g., antigen-binding.

In certain embodiments, antibody variants and/or binding polypeptide variants having one or more amino acid substitutions are provided. Sites of interest for substitutional mutagenesis include the HVRs and FRs. Conservative substitutions are shown in Table 1 under the heading of “preferred substitutions.” More substantial changes are provided in Table 1 under the heading of “exemplary substitutions,” and as further described below in reference to amino acid side chain classes. Amino acid substitutions may be introduced into an antibody and/or binding polypeptide of interest and the products screened for a desired activity, e.g., retained/improved antigen binding, decreased immunogenicity, or improved ADCC or CDC.

TABLE 1

| | | Preferred |
|---|---|---|
| Original Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g., binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or residues that contact antigen with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen contacting residues in the HVRs. In certain embodiments of the variant VH and VL sequences provided above, each HVR either is unaltered, or contains no more than one, two or three amino acid substitutions.

A useful method for identification of residues or regions of the antibody and/or the binding polypeptide that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

All patent and literature references cited in the present specification are hereby incorporated by reference in their entirety.

### EXAMPLES

The following are examples of methods and compositions of the invention. It is understood that various other embodiments may be practiced, given the general description provided above.

### Example 1

### Organoids Grown in Liquid Culture

The growth of organoid cultures from various organ tissue fragments has been demonstrated. The following is a technical description to allow organoid growth from tissue fragments comprising stem cells (e.g., small intestine) while maximizing the feasibility of performing downstream applications aimed at understanding the biology of organoids.

### Materials and Methods

Mice

CD-1 outbred mice between the ages of 6 and 12 weeks were used for crypt isolation.

Crypt Isolation

The small intestine comprising the duodenum, jejunum, and ileum was harvested from a single mouse for individual plating experiments. The small intestine was flushed once with cold PBS and opened longitudinally. A cell scraper was used to remove villus structures, thus exposing crypt structures to working solutions. The intestine was then chopped into approximately 5 mm pieces and incubated in cold chelation buffer (5.6 mM Na₂HPO₄, 96.2 mM NaCl, 1.6 mM KCl, 43.4 mM Sucrose, 54.9 D-Sorbitol) plus 2 mM EDTA, 0.5M DL-Dithiothereitol for 30 minutes on ice. The chelation EDTA-DTT buffer was removed and tissue fragments were vigorously re-suspended in cold chelation buffer using a 10-mL pipette. The appearance of free crypts in solution was monitored using a dissecting microscope. The process of incubation in chelation EDTA-DTT buffer and re-suspension was repeated until free crypts reached a concentration of 10 crypts/μL, usually after 3 cycles. Supernatant containing crypts was collected in 15 mL conical tubes, pelleted at 150-200 g for 3 minutes and washed with cold chelation buffer.

Plating and Culturing

After washing in chelation buffer, crypts were pelleted again and re-suspended in growth media (see below) at a concentration of 5 crypts/μL. The crypts were then plated on GravityPLUS 96-well hanging-drop plates (inSphero) at 40 μL (200 crypts) per well. 10 mL of PBS was added to the bottom of the plate to prevent evaporation and plates were cultured in a humidified incubator at 37° C., 5% CO₂, and atmospheric oxygen. Every 2 days, half of the media was removed from each well and replaced with fresh media to ensure a constant source of factors required for growth.

Proliferation and Differentiation Assay

After 10 days of culturing, the plates were coupled to GravityPLUS receiver plates (InSphero) and centrifuged at 150-200 g for 3 minutes. The media was removed with a multi-channel pipette and the organoids were incubated in growth media plus 10 μM EdU at 37° C. for 30 minutes. The organoids were then fixed in 4% paraformaldehyde/PBS for 15 minutes at room temperature. The organoids were washed 2× in PBS plus 3% BSA and then permeabilized in PBS plus 0.5% Trion X-100 at room temperature for 20 minutes. After permeabilization, the organoids were incubated in Click-iT EdU reaction cocktail and processed according to manufacturer's protocol (Invitrogen). Organoids were washed 2× with PBS plus 0.1% Trion X-100 and incubated in rabbit anti-human Lysozyme primary antibody at 1:3000 overnight at 4° C. The following day, the organoids were washed 2× with PBS plus 0.1% Trion X-100, incubated in alexa-fluor 488 secondary for 1 hour at room temperature, washed 2× with PBS plus 0.1% Trion X-100, mounted in prolong gold mounting media and imaged on a Leica SPE confocal microscope.

Growth Media

Advanced DMEM/F12 supplemented with penicillin/streptomycin, 1×N2 (Gibco), 1×B27 (Gibco), 10 mM HEPES, 1× Glutamax (Gibco), 1 mM N-acetylcysteine, murine EGF 10-50 ng/mL (PeproTech), murine Noggin 50-100 ng/mL (PeproTech), hRSPO3 1 μg/mL, and 0-5% Growth factor reduced Matrigel (BD Biosciences).

### Results

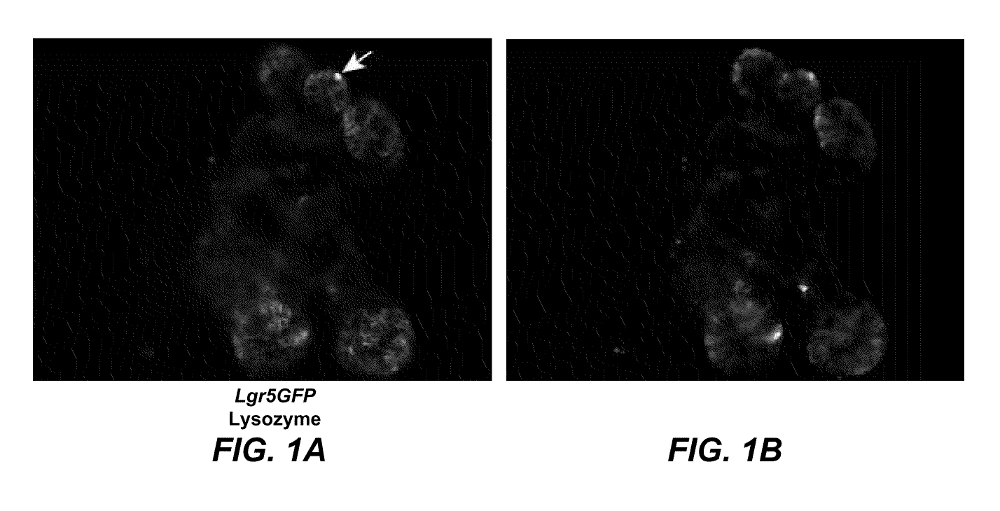

Consistent organoid growth was observed using the method described above with 5% matrigel. The presence and location of GFP staining in the crypt-like structures that bud off from the main organoid body was consistent with the localization of Lgr5 expression at the base of the crypt in vivo (FIG. 1). In addition, lysozyme staining in the crypt-like structures of the organoids was observed, suggesting that this preparation led to organoids that actively differentiate specialized cells of the gut (FIG. 1).

### Example 2

### Ablation of Lgr5 Positive Stem Cells in the Gut Organoids

Previous studies have shown that ablation of Lgr5 positive stem cells in the murine gut resulted in normal gut morphology as well as a normal distribution and number of differentiated cells (Tian et al 2011). To determine whether the liquid cultured organoids mimic the functional and morphological characteristic of in vivo gut tissue, Lgr5 positive stem cells were ablated in liquid cultured organoids as described below.

### Materials and Methods

Mice

Lgr5^{DTREGFP/+} mice originally characterized in Tian et al., 2011 were used to isolate crypts as above. These mice serve as reporters for Lgr5 expression utilizing an EGFP cassette and allow for Lgr5 positive stem cell ablation upon administration of Diptheria Toxin (DT).

Lgr5 Positive Stem Cell Ablation

Crypt isolation, plating, and culturing were performed as above except in the case of Lgr5 positive stem cell ablation where crypts were grown for 3-7 days and visually inspected for the presence of organoids. Organoids were then treated with media containing 10 ng/mL Diptheria Toxin (DT). The media containing DT and media without DT for control organoids was replaced every two days for a total of 10 days.

Organoid Staining

Treated and control organoids were then harvested and stained for GFP to monitor the effects of Lgr5 stem cell ablation. Simultaneously, organoids were co-stained for Lysozyme to identify differentiated Paneth cells. Organoids were washed 2× with PBS plus 0.1% Trion X-100, fixed in 4% paraformaldehyde/PBS for 15 minutes at room temperature. The organoids were washed 3× in PBS plus 0.1% Trion X-100 and blocked with protein-free block (Dako) for 1 hour. The organoids were incubated with chicken anti-GFP (1:2000) and rabbit anti-human Lysozyme (1:2000) antibodies overnight at 4° C. The following day, the organoids were washed 3× with PBS plus 0.1% Trion X-100, incubated in anti-chicken IgG Cy3 ( ) and anti-rabbit alexa-fluor 488 secondaries for 1 hour at room temperature, washed 2× with PBS plus 0.1% Trion X-100, mounted in prolong gold mounting media and imaged on a Leica SPE confocal microscope.

### Results

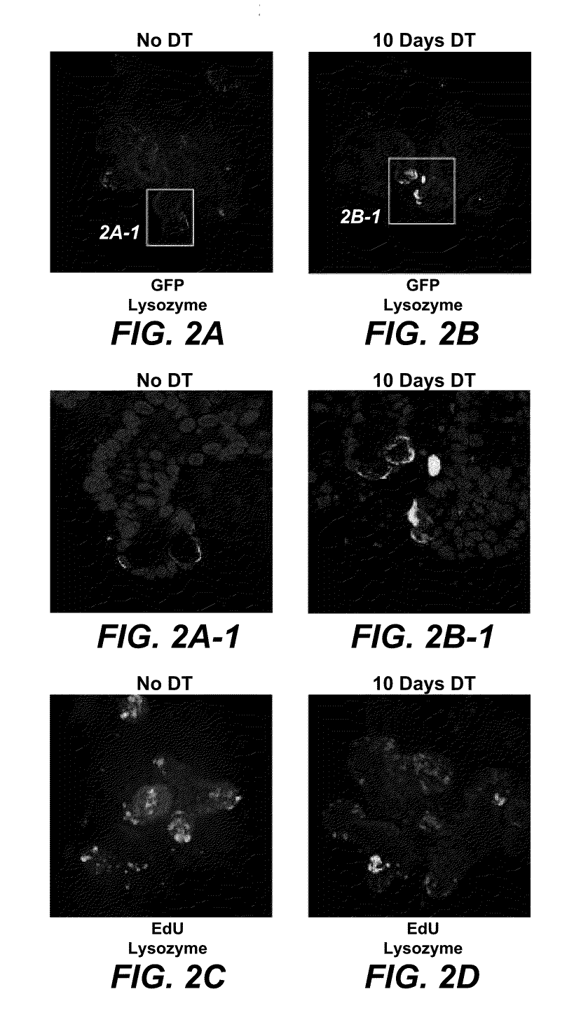

Long-term incubation of Lgr5^{DTREGFP/+} organoids in the presence of DT resulted in normal appearing organoids that lacked any Lgr5 dependent GFP expression due to ablation of Lgr5 expressing cells (FIG. 2b). These organoids were positive for Lysozyme staining indicating that differentiation occurred in the absence of Lgr5 positive cells (green staining, FIGS. 2b, b′ and d) and showed robust proliferation (red staining, FIG. 2d).

In summary, a single intestinal stem cell was shown to be able to operate independently of positional cues from its environment including a solid extracellular matrix and that it can generate a continuously expanding, self-organizing epithelial structure reminiscent of normal gut. The described culture system comprising liquid culturing methods will simplify the study of stem cell-driven crypt-villus biology.

### Example 3

### The Requirement of ECM (Matrigel) for Gut Organoid Growth

To determine the requirement of Matrigel on gut organoid growth, organoids were grown using the hanging drop method with different concentrations of Matrigel in the growth media.

### Mice, Organoid Preparation, and Organoid Staining

WT CD-1 mice were used for these experiments. Crypt isolation, plating, and culturing were performed as above except organoids were grown in the presence of 0%, 1%, 2%, 3%, 4%, and 5% Matrigel. Plates containing organoids were visually inspected 7 days post crypt seeding. Organoids were stained and imaged as described above.

### Results

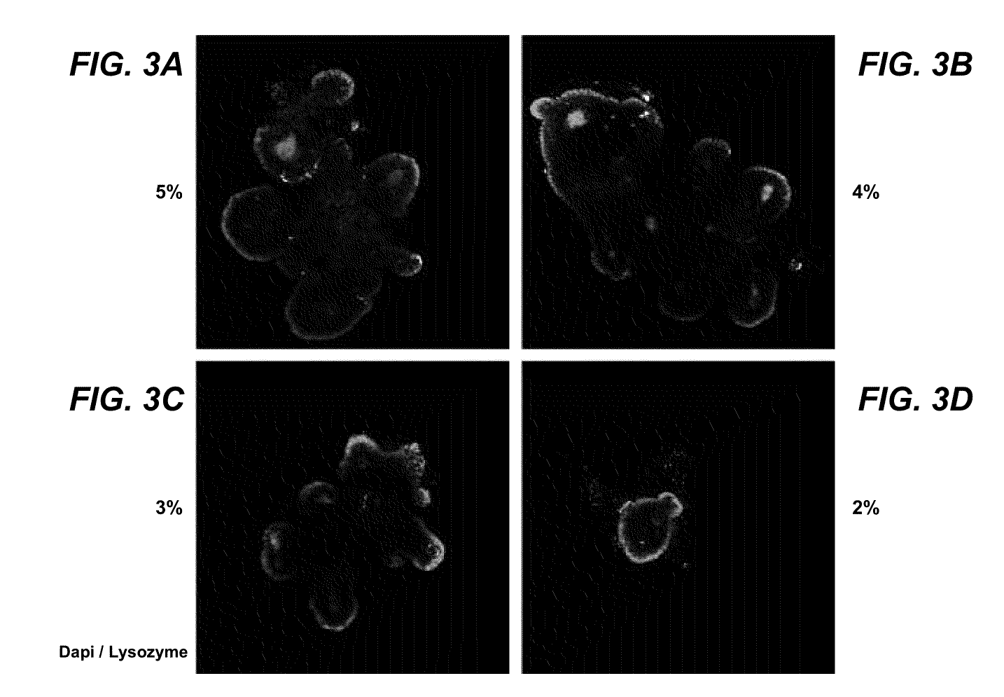

FIG. 3 shows the results of crypts seeded with diminishing concentrations of Matrigel in the growth media. 5% and 4% Matrigel (a and b, respectively) produced organoids of consistently large size. Lower concentrations of Matrigel did not support robust growth of organoinds (c and d) with 1% and 0% Matrigel showing no growth whatsoever (data not shown).

1: A method for liquid culturing (a) epithelial stem cells and/or (b) isolated epithelial tissue fragments comprising epithelial stem cells, the method comprising incubating the epithelial stem cells and/or the isolated tissue fragments in a liquid cell culture comprising a basal medium for animal or human cells to which is added (i) a Bone Morphogenetic Protein (BMP) inhibitor, (ii) a mitogenic growth factor, (iii) Wnt agonist, and (iv) at least about 4% w/v of extracellular matrix (ECM).

2: A method for obtaining and/or growing a crypt, the method comprising incubating epithelial stem cells and/or isolated tissue fragments in a liquid cell culture comprising a basal medium for animal or human cells to which is added (i) a Bone Morphogenetic Protein (BMP) inhibitor, (ii) a mitogenic growth factor, (iii) Wnt agonist, and (iv) at least about 4% w/v of extracellular matrix (ECM).

3: The method of claim 1, wherein the BMP inhibitor is Noggin, DAN, and/or DAN-like proteins including Cerberus and Gremlin.

4: The method of claim 3, wherein the BMP inhibitor is Noggin.

5: The method of claim 1, wherein the BMP inhibitor is at a concentration between about 5 and about 500 ng/ml in the liquid cell culture (e.g., about 50 to about 100 ng/mL).

6: The method of claim 1, wherein the Wnt agonist is a Wnt, an R-spondin (RSPO), Norrin, and/or a GSK-inhibitor.

7. The method of claim 6, wherein the Wnt agonist is RSPO.

8: The method of claim 1, wherein the Wnt agonist is at a concentration between about 500 ng/mL and about 5 μg/ml in the liquid cell culture (e.g., about 500 to about 1500 ng/mL).

9: The method of claim 1, wherein the mitogenic growth factor is epidermal growth factor (EGF), Transforming Growth Factor-alpha (TGF-α), basic Fibroblast Growth Factor (bFGF), brain-derived neurotrophic factor (BDNF), and Keratinocyte Growth Factor (KGF).

10. The method of claim 9, wherein the mitogenic growth factor is EGF.

11. The method of claim 1, wherein the mitogenic growth factor is at a concentration between about 5 and about 500 ng/ml in the liquid cell culture (e.g., about 5 to about 50 ng/mL).

12. The method of claim 1, wherein the ECM is a growth factor reduced ECM.

13. The method of claim 1, wherein the ECM is matrigel.

14. The method of claim 1, wherein the ECM is at a concentration of between about 4% to about 10% w/v in the liquid cell culture.

15. The method of claim 1, wherein the culture medium further comprises a Rock (Rho-kinase) inhibitor.

16. The method of claim 1, wherein the culture medium further comprises a Notch agonist.

17. The method of claim 1, wherein the epithelial stem cells and/or epithelial tissue fragments are gastrointestinal stem cells and/or gastrointestinal tissue fragments.

18. The method of claim 17, wherein the gastrointestinal stem cells and/or gastrointestinal tissue fragments are small intestine stem cells and/or small intestine tissue fragments.

19. A crypt obtainable by the methods of claim 1.

20. Use of the crypt of claim 19 in a drug discovery screen, toxicity assay, or in regenerative medicine.
